(19) United States
(12) Patent Application Publication
(10) Pub. No.: US 2011/0112469 A1
(43) Pub. Date: May 12, 2011

(54) DEVICE AND PROCESS FOR DISPENSING MULTIPLE-PHASE MIXTURES

(75) Inventors: Amir A. Naqwi, Eden Prairie, MN (US); Zeeshan H. Syedain, Minneapolis, MN (US)

(73) Assignee: POWERSCOPE INCORPORATED, Minneapolis, MN (US)

(21) Appl. No.: 12/940,325

(22) Filed: Nov. 5, 2010

Related U.S. Application Data
(60) Provisional application No. 61/259,204, filed on Nov. 8, 2009.

(51) Int. Cl. A61M 13/00

(52) U.S. Cl. 604/24

(57) ABSTRACT

A device and method for delivering a multi-phase mixture of a composition dispersed in a gas, the device comprising an outer needle having an inlet opening configured to receive a supply of the composition, and an inner needle disposed within the outer needle, the inner needle comprising an inlet opening configured to receive a pressurized supply of the gas, where a tip of the inner needle is offset inward from a tip of the outer needle along a longitudinal axis by an offset distance.

### CROSS-REFERENCE TO RELATED APPLICATION(S)

The present application claims priority to U.S. Provisional Patent Application No. 61/259,204, filed on Nov. 8, 2009, and entitled “DEVICE AND PROCESS FOR DISPERSION OF MULTIPHASE MIXTURES THROUGH A NEEDLE”, the disclosure of which is incorporated by reference in its entirety.

### STATEMENT OF GOVERNMENT INTEREST

This invention was made with government support under Contract No. R44EY016229 awarded by the National Eye Institute, National Institutes of Health. The government has certain rights in the invention.

### BACKGROUND

The present disclosure relates to the formation and dispensing of multiple-phase mixtures. In particular, the present disclosure relates to needle-based devices and processes for dispensing multiple-phase mixtures, such as for use in insufflation procedures.

Insufflation is a common medical procedure to expand operating space within a body cavity, or to minimize obstructions during minimally invasive or laparoscopic surgeries. Such procedures involve introducing an inert gas within the body cavity at a suitable pressure to expand the body cavity, while reducing the risk of ruptures.

The inert gases are typically introduced into the body cavity through a gas-insufflation needle device. Prior to insufflation, the needle device is inserted through an opening in the body cavity. The gas is then introduced into the cavity and may be vented through another device to maintain a suitable pressure within the cavity. Modifications to these needle devices have been made to increase the patient safety and ease of use. For example, components for heating the inert gases have been included, as well as movable parts for a better control over the amount of the delivered gas and pressure of the gas in the insufflated cavity.

Additional developments have addressed delivery of materials to specific organs, such as eyes (e.g., for laparoscopic surgeries), lungs, and the like. These devices, however, lack features for delivering of inhomogeneous, multi-phase mixtures. For example, a suspension of droplets in air may not be effectively delivered using these devices, as the droplets condensate in the needle and may obstruct the flow. As such, there is an ongoing need for devices and processes for delivering multi-phase mixtures to confined spaces, such as for surgical procedures.

### SUMMARY

A first aspect of the present disclosure is directed to a device for delivering a multi-phase mixture of a composition dispersed in a gas. The device includes an outer needle having a hub region and a tip separated along a longitudinal axis, where the hub region of the outer needle includes an inlet opening configured to receive a supply of the composition. The device also includes an inner needle disposed within the outer needle, where the inner needle has a hub region and a tip separated along the longitudinal axis, and where the hub region of the inner needle includes an inlet opening configured to receive a pressurized supply of the gas. The tip of the inner needle is offset inward from the tip of the outer needle along the longitudinal axis by an offset distance.

Another aspect of the present disclosure is directed to a device for delivering a multi-phase mixture of a composition dispersed in a gas, where the device includes a base component configured to receive a supply of the gas, and a needle assembly connectable to the base component. The needle assembly includes an outer needle having an inlet opening adjacent to the base component and configured to receive a supply of the composition, and a tip. The needle assembly also includes an inner needle disposed within the outer needle, where the inner needle has an inlet opening configured to receive the gas from the base component, and a tip. The tip of the inner needle is offset inward from the tip of the outer needle along a longitudinal axis of the needle assembly by an offset distance.

Another aspect of the present disclosure is directed to a method for dispensing a multi-phase mixture. The method includes providing a needle assembly having an outer needle and an inner needle disposed within outer needle, where the needle assembly has a proximal end and a distal end offset along a longitudinal axis, and where the outer needle and the inner needle each have a tip at the distal end of the needle assembly. The method also includes directing pressurized gas through an inner lumen of the inner needle such that the pressurized gas is emitted from the tip of the inner needle. The emission of the pressurized gas from the tip of the inner needle generates a vacuum in a lumen region between the outer needle and the inner needle, which draws a composition through the lumen region between the outer needle and the inner needle. The method further includes mixing the drawn composition and the emitted pressurized gas in a dispersion region, which is located along the longitudinal axis between the tip of the inner needle and the tip of the outer needle, to form the multi-phase mixture, and ejecting the multi-phase mixture from the distal end of the needle assembly.

### BRIEF DESCRIPTION OF THE DRAWINGS

FIG. 1 is a schematic view of a device of the present disclosure inserted within an eye for assisting surgical procedure called vitrectomy.

FIG. 2 is a side schematic illustration of a suitable embodiment of the device of the present disclosure.

FIG. 3 is an expanded and broken view a needle assembly of the device connected to a base portion of the device.

FIG. 4 is a photograph of a distal end of the needle assembly of the device, depicting a multi-phase mixture being dispensed.

### DETAILED DESCRIPTION

The present disclosure is directed to a needle-based device for dispensing multi-phase mixtures to confined spaces. As discussed below, the device of the present disclosure includes multiple concentric needles for supplying individual components of a multi-phase mixture (e.g., gases, liquids, and solid particles) to the tips or distal ends of the needles. Upon reaching the distal ends, the individual components mix together in a non-homogenous manner to form the multi-phase mixture. This mixing of the individual components at the distal ends of the needles allows the multi-phase mixture to remain non-homogenous as the mixture is dispensed to the desired target location. As such, the needle-based device is suitable for minimally-invasive, drug delivery applications to various cavities and locations within a human or animal body, such as for vitrectomy and laparoscopic surgeries.

As used herein, the term “multi-phase mixture” refers to a mixture of one or more compositions that are suspended in a carrier medium in a non-homogenous manner. Examples of suitable multi-phase mixtures include aerosolized liquid droplets that are suspended in a gas, aerosolized solid particles suspended in a gas, and combinations thereof. The following discussion of the liquid and solid particles is made with reference to medical drugs for use in surgical procedures. However, the needle-based device of the present disclosure may be used to form multi-phase mixtures having a variety of different compositions suspended in a gas, where the particular compositions and gases may vary depending on the intended applications.

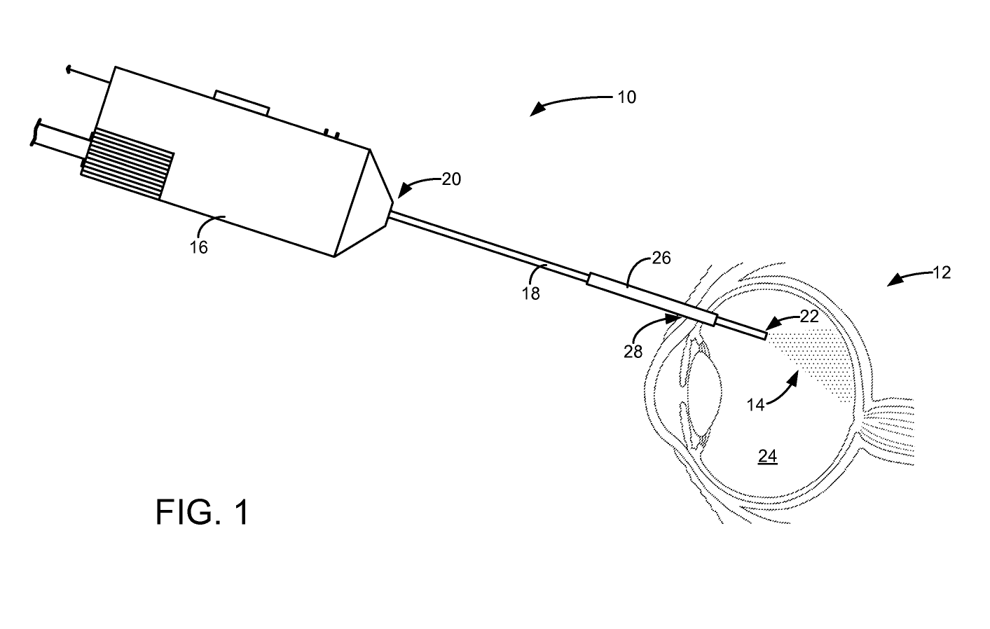

FIG. 1 illustrates device 10 inserted into eye 12, where device 10 is an example of a suitable needle-based device of the present disclosure for delivering an aerosol spray of a multi-phase mixture, referred to as spray 14, to a target location within eye 12. Accordingly, device 10 is suitable for use in vitrectomy-based procedures to dispense one or more drugs in spray 14, such as anti-scarring drugs, glaucoma-treatment drugs (e.g., mitomycin C), and the like.

Device 10 includes base portion 16 and needle assembly 18, where needle assembly 18 includes proximal end 20 operably connected to base portion 16, and tip or distal end 22 extending within interior cavity 24 of eye 12. As further shown in FIG. 1, needle assembly 18 extends through cannula 26, which is inserted through opening 28 in the sclera and choroid of eye 12. Cannula 26 may have any suitable size, such as dimensions ranging from gauge 22 to gauge 25. During use in a vitrectomy-based procedure, opening 28 is formed in eye 12 and cannula 26 is inserted through opening 28 to provide access to the interior cavity 24. Needle assembly 18 is then inserted through cannula 26 such that distal end 22 extends within interior cavity 24.

Typically, one or more additional openings are made in eye 12 to provide access for other devices (not shown), such as a light source and a vacuum line. In some ocular surgical procedures, the vitreous humor may be removed from interior cavity 24 through the vacuum line, and pressurized gas (e.g., pressurized air) may be introduced through base portion 16 and needle assembly 18 into interior cavity 24. A constant pressure may be maintained within interior cavity 24 by balancing the input and removal of the pressurized gas. The pressurized gas within interior cavity 24 maintains a positive pressure within eye 12, which holds the retina in place and minimizes the reintroduction of fluids during surgery.

After surgery is complete, a drug may be supplied to base portion 16 as a liquid and/or as solid particles. The supplied drug is then drawn from base portion 16 into needle assembly 18 due to the pressurized gas flow through needle assembly 18. As discussed below, needle assembly 18 includes multiple concentric lumens or needle pathways. A first pathway of needle assembly 18 is configured to direct the flow of the pressurized gas from base portion 16 to distal end 22. A second pathway of needle assembly 18 is configured to draw the supplied drug from base portion 12 to distal end 22 via a venturi effect from the flow of the pressurized gas in a similar manner to an aspirator.

At distal end 22, the drug mixes with the flow of the pressurized gas to form a multi-phase mixture of the drug suspended in the gas. The resulting multi-phase mixture is then ejected to a target location within interior cavity 24 as spray 14, where spray 14 may have a conical region with a vertex angle of at least about 20 degrees, for example. This arrangement allows the multi-phase mixture to be formed at distal end 22, rather than at an upstream location along needle assembly 18 or within base component 16. The formation of the multi-phase mixture at distal end 22 accordingly allows the liquid droplets and/or solid particles of the drug to remain suspended within the emitted gas, thereby increasing control of applying the drug to a desired target location within interior cavity 24. For example, the multi-phase mixture may include aerosolized liquid droplets suspended in the gas, where the liquid droplets contain one or more drugs to prevent the growth of scar tissues within eye 12.

The use of device 10 in this manner also allows the same device that supplies the pressurized gas within a body cavity to also supply a variety of different drugs to target sites within the body cavity. This accordingly reduces the number of devices that are required to be inserted within the walls of the body cavity, and also reduces time required to perform the drug deliveries. As such, device 10 may be used to deliver aerosolized drugs to insufflated cavities during a variety of different laparoscopic procedures.

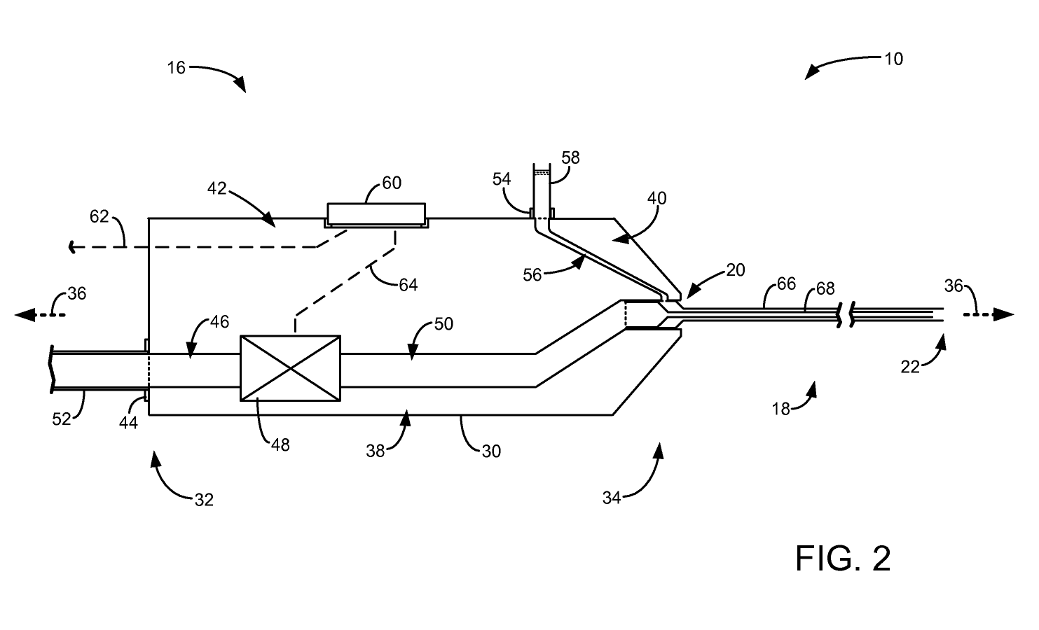

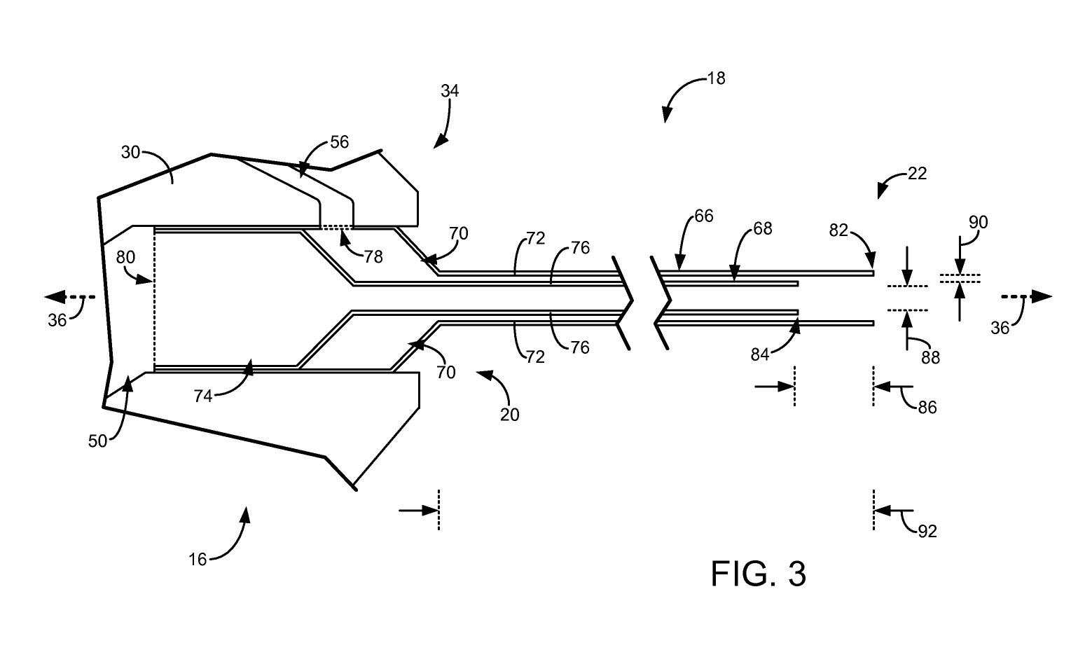

FIGS. 2 and 3 illustrate a suitable embodiment of device 10 that includes base portion 16 and needle assembly 18. As shown in FIG. 2, base portion 16 includes housing 30, which has foot end 32 and head end 34 offset along longitudinal axis 36, where proximal end 20 of needle assembly 18 is connected to head end 34 of housing 30. Housing 30 may be fabricated from a variety of different materials, such as medical-grade plastics and metals, which are desirably autoclavable. Housing 30 may also be designed as a hand-held component, having an outer-surface geometry that is easily graspable and handled by a user during a surgical procedure.

Base portion 16 also includes gas line 38, drug line 40, and electrical system 42, which are retained at least partially within housing 30. Gas line 38 includes coupling 44, intake conduit 46, valve 48, and manifold conduit 50, where coupling 44 is located at an outer surface of housing 30 and is configured to engage with an external gas line, such as gas line 52. This arrangement allows pressurized gas from gas line 52 to be delivered to needle assembly 18 through the operation of valve 48. Accordingly, valve 48 may be any suitable valve assembly operable with electrical system 42, such as a solenoid valve.

Drug line 40 includes coupling 54 and flow port 56. Coupling 54 is located at the outer surface of housing 30 and is configured to engage with an external supply source of the drug to be delivered. For example, as shown in FIG. 2, inlet coupling 54 may be configured to engage with capsule 58, which is a disposable, single-dose capsule containing a liquid or solid-particle drug that may be removably secured to coupling 54. This arrangement allows the drug from capsule 58 to be drawn through flow port 56 to needle assembly 18 via a venturi effect from the flow of the pressurized gas through needle assembly 18, as discussed below. The inlet of flow port 56 adjacent to coupling 54 is desirably sealable when capsule 58 is not engaged with coupling 54, thereby preventing ambient air from being drawn through flow port 56 and needle assembly 18 during use.

In an alternative embodiment, inlet coupling 54 may be configured to engage with a feed line (not shown) of the liquid or solid-particle drug rather than single-dose capsule 58. In this embodiment, the feed line may interconnect base portion 16 and an external source (not shown) of the liquid or solid-particle drug. The liquid or solid-particle drug may accordingly be drawn through flow port 56 and needle assembly 18 via the venturi effect, or may be actively pumped through flow port 56 and needle assembly 18 with an external pump system (not shown).

In additional alternative embodiments, drug line 40 may be located at different locations along housing 30. For example, coupling 54 may alternatively be located adjacent to foot end 32 of housing 30, and flow port 56 may extend through housing 30 between coupling 54 and needle assembly 18. In further alternative embodiments, drug line 40 may be located external to housing 30. For example, needle assembly 18 may include a coupling corresponding to coupling 54 to directly receive a supply of the drug, such as with capsule 58.

Electrical system 42 includes switch 60 and electrical lines 62 and 64. In the shown embodiment, switch 60 is a button-actuated contact switch that opens and closes the electrical connection between electrical lines 62 and 64. Electrical line 62 extends out of housing 30 and interconnects switch 60 with an external power source (not shown). In one embodiment, gas line 52 and electrical line 62 interconnect base portion 12 of device 10 with a remote unit that supplies the pressurized gas and electrical power to base portion 12. For example, gas line 52 and electrical line 62 maybe connected to a vision surgery system, such as the vision systems commercially available from Alcon, Inc., Hunenberg, Switzerland, thereby allowing device 10 to be integrated into to a variety of existing vision systems. Electrical line 64 interconnects valve 48 and switch 60, which allows valve 48 to be opened by the actuation of switch 60.

In an alternative embodiment, switch 60 may be omitted, and the operation of valve 48 may be performed with a switch that is located outside of housing 30 at a remote location, such as with a foot-pedal switch. In this embodiment, electrical line 62 may connect valve 48 to the external and remote switch.

As further shown in FIG. 2, needle assembly 18 includes outer needle 66 and inner needle 68, where inner needle 68 extends concentrically within outer needle 66 along longitudinal axis 36. Outer needle 66 and inner needle 68 may each be fabricated from a variety of different materials, such as medical-grade plastics and metals, which are also desirably autoclavable.

In the shown example, proximal end 20 of needle assembly 18 extends within head end 34 of housing 30 to engage with base portion 16. Proximal end 20 of needle assembly 18 may engage with and be connected to base portion 16 using a variety of different engagement mechanisms. For example, proximal end 20 of needle assembly 18 may be operably connected to head end 34 of housing 30 using a threaded engagement, a frictional fit, a locking mechanism, an adhesive composition, and combinations thereof. Alternatively, needle assembly 18 may engage with head end 34 of housing 30 at an external location of housing 30 (rather than extending within head end 34). Furthermore, in one embodiment, the operable connection between base portion 16 and needle assembly 18 allows needle assembly 18 to be removably connected to base portion 16, thereby allowing multiple needle assemblies 18 to be interchangeably connected to base portion 16.

During operation, a user may grip and manipulate base portion 16 to insert needle assembly 18 into a body cavity, such as through cannula 26 into interior cavity 24 of eye 12 (shown in FIG. 1), as discussed above. The user may then actuate switch 60 to close the circuit between electrical lines 62 and 64. This allows electrical power to flow to valve 48, thereby opening valve 48. The pressurized gas from gas line 52 then flows through intake conduit 46, valve 48, and manifold conduit 50 to inner needle 68. The pressurized gas flows through the inner lumen or pathway of inner needle 68 and exits needle assembly 18 at distal end 22 to fill interior cavity 24 to a steady-state pressure, as discussed above. Suitable introduced gas pressures for use with device 10 range from about 10 pounds/square-inch to about 60 pounds/square-inch. The user may then perform surgery on eye 12 while the gas continues to flow through interior cavity 24.

When the surgery is complete, the user may then mount capsule 58, containing a liquid and/or solid-particle drug, to inlet coupling 54. The continuous flow of the gas through inner needle 68 draws the drug through flow port 56 and the annular lumen region or pathway between outer needle 66 and inner needle 68 toward distal end 22, via a venturi effect. The venturi effect draws the drug through needle assembly 18 at a substantially slower flow rate than the flow rate of the gas through needle assembly 18. As discussed below, this difference in flow rates, along with the dimensions of distal end 22, nebulizes the drawn drug in the flow of gas, thereby rendering the drug aerosol at distal end 22.

For example, in embodiments in which the drug is provided as a liquid, the liquid is broken down into small droplets at distal end 22, which are suspended in the flow of the gas to provide the multi-phase mixture of spray 14 (shown in FIG. 1). The formation of the multi-phase mixture at distal end 22 accordingly allows the liquid droplets and/or solid particles of the drug to remain suspended within the gas, thereby increasing the control of applying the drug to a desired target location.

FIG. 3 is an expanded and broken view of needle assembly 18 engaged with base portion 16. As shown, outer needle 66 includes hub region 70 and shaft 72, where hub region 70 is located at proximal end 20 of needle assembly 18, and shaft 72 extends along longitudinal axis 36 between hub region 70 and distal end 22. Correspondingly, inner needle 68 includes hub region 74 and shaft 76, where hub region 74 is located at proximal end 20 of needle assembly 18, and shaft 76 extends along longitudinal axis 36, within the lumen of shaft 72, between hub region 74 and distal end 22.

In the shown embodiment, hub region 70 includes lateral opening 78, and hub region 74 includes axial opening 80. Lateral opening 78 is an opening in outer needle 66 that is accessible in a direction that is orthogonal to longitudinal axis 36, and axial opening 80 is an opening in inner needle 68 that is parallel (e.g., co-linear) with longitudinal axis 36. As such, when needle assembly 18 is connected to housing 30, lateral opening 78 of hub region 70 accesses flow port 56, and axial opening 80 of hub region 74 accesses manifold conduit 50.

During operation, the pressurized gas flowing through manifold conduit 50 enters hub region 74 via axial opening 80. As shown, hub region 74 converges from a first diameter at axial opening 80 down to a second, narrower diameter at shaft 76, which increases the flow rate of the gas through the lumen of shaft 76. As the high-speed gas flows out of shaft 76, the flow generates a vacuum through the annular lumen region between shafts 72 and 76 via a venturi effect. This vacuum draws the liquid or solid particles of the drug through flow port 56, lateral opening 78, hub region 74, and the annular lumen region between shafts 72 and 76.

At distal end 22, shaft 72 of outer needle 66 includes tip 82, and shaft 76 of inner needle 68 includes tip 84, where tip 84 is offset inward from tip 82 to define a dispersion region in which the drug and gas mix. The dimensions of outer needle 66, inner needle 68, and the fixed offset distance between tips 82 and 84 (referred to as offset distance 86) are set to provide a suitable level of nebulization of the drug in the gas flow. In particular, for particular flow rates of the gas and drug through shafts 72 and 76, offset distance 86 is desirably balanced to suspend the droplets or particles in the gas flow. If offset distance 86 is too short, the gas and drug do not have enough residence time at distal end 22 to interact enough to obtain effective nebulization. On the other hand, if offset distance 86 is too long, the liquid droplets or solid particles begin to coalesce into larger drops or particles before exiting distal end 22.

Examples of suitable distances for offset distance 86 range from about 0.01 millimeters to about 1.0 millimeter, with particularly suitable distances ranging from about 0.1 millimeters to about 0.5 millimeters, and with even more particularly suitable distances ranging from about 0.2 millimeters to about 0.4 millimeters. The terms “about” and “substantially” are used herein with respect to measurable values and ranges due to expected variations known to those skilled in the art (e.g., limitations and variabilities in measurements).

A suitable distance for offset distance 86 may be selected in combination with the dimension of outer needle 66 and inner needle 68. In particular, offset distance 86 may be selected based on the inner diameter of inner needle 68 (referred to as diameter 88), and on the dimensions of the annular lumen region outer needle 66 and inner needle 68 (referred to as annular distance 90).

Examples of suitable average distances for diameter 88 range from about 0.05 millimeters to about 0.5 millimeters, with particularly suitable average distances ranging from about 0.1 millimeters to about 0.3 millimeters, and with even more particularly suitable average distances ranging from about 0.1 millimeters to about 0.2 millimeters. These distances for diameter 88 are suitable for providing gas flow rates through inner needle 68 ranging from about 0.02 liters/minute to about 2.0 liters/minute. Examples of suitable average distances for annular distance 90 range from about 0.01 millimeters to about 0.15 millimeters, with particularly suitable average distances ranging from about 0.01 millimeters to about 0.1 millimeters, and with even more particularly suitable average distances ranging from about 0.01 millimeters to about 0.05 millimeters.

Examples of suitable combinations of offset distance 86, diameter 88, and annular distance 90, for introduced gas pressures ranging from about 10 pounds/square-inch to about 60 pounds/square-inch, include offset distance 86 ranging from about 0.2 millimeters to about 0.4 millimeters, diameter 88 ranging from about 0.1 millimeters to about 0.2 millimeters, and annular distance 90 ranging from about 0.01 millimeters to about 0.05 millimeters.

While outer needle 66 and inner needle 68 are discussed herein as having cylindrical geometries, in alternative embodiments, one or both of outer needle 66 and inner needle 68 may have non-cylindrical geometries (e.g., oval or rectangular geometries). In additional alternative embodiments, inner needle 68 is not located concentrically within outer needle 66, and may have a central axis that is not co-linear with a central axis of outer needle 66. Accordingly, in these embodiments, the dimensions of outer needle 66 and inner needle 68 may be determined by their cross-sectional areas rather than their respective diameters. The cross-sectional areas of the needles discussed herein are taken in a plane that is orthogonal to the longitudinal axis of the needles (e.g., longitudinal axis 36).

As such, examples of suitable average cross-sectional areas for the inner lumen of inner needle 68 (corresponding to diameter 88) range from about 0.002 square millimeters to about 0.2 square millimeters, with particularly suitable average cross-sectional areas ranging from about 0.008 square millimeters to about 0.07 square millimeters, and with even more particularly suitable average cross-sectional areas ranging from about 0.008 square millimeters to about 0.03 square millimeters.

Examples of suitable average cross-sectional areas for the lumen region between the inner dimensions of outer needle 66 and the outer dimensions of inner needle 68 (corresponding to annular distance 90) range from about 0.005 square millimeters to about 0.14 square millimeters, with particularly suitable average cross-sectional areas ranging from about 0.005 square millimeters to about 0.08 square millimeters, and with even more particularly suitable average cross-sectional areas ranging from about 0.005 square millimeters to about 0.03 square millimeters.

Suitable lengths for shaft 72 of outer needle 66 (referred to as length 92) may vary depending on the particular intended use of device 10. Examples of suitable average distances for length 92 range from about 5 millimeters to about 50 millimeters. As shown, tip 82 of outer needle 66 and tip 84 of inner needle 68 are desirably blunt tips that are fixed relative to each other at offset distance 86. This provides a suitable level of nebulization of the drug in the gas flow at the dispersion region of needle assembly 18.

As discussed above, during operation, pressurized gas may flow through the lumen of inner needle 68. When the drug is to be delivered, the user may then mount capsule 58, containing a liquid and/or solid-particle drug, to inlet coupling 54 (shown in FIG. 2). The continuous flow of the gas through inner needle 68 draws the drug through flow port 56 and through the lumen region between outer needle 66 and inner needle 68 toward distal end 22, via a venturi effect. The venturi effect draws the drug through the lumen between outer needle 66 and inner needle 68 at a substantially slower flow rate than the flow rate of the gas through the lumen of inner needle 68.

When the drug reaches the dispersion region between tips 82 and 84, the offset distance 86 between tips 82 and 84 allows the drawn drug to nebulize in the flow of gas, thereby rendering the drug aerosol at distal end 22 to provide a multi-phase mixture (e.g., spray 14, shown in FIG. 1). As discussed above, the formation of the multi-phase mixture at distal end 22 allows the drug to remain suspended within the gas, thereby increasing the control of applying the drug to a desired target location.

Suitable drugs for use with device 10 may vary depending on the intended use. Examples of suitable drugs for use with device 10 include anti-scarring drugs, glaucoma-treatment drugs (e.g., mitomycin C), cancer-treatment drugs (e.g., Fluorouracil, 5-FU), anti-oxidants (e.g., melatonin), lung surfactant proteins (e.g., an intratracheal suspension under the trade designation “CUROSURF” from Cornerstone Therapeutics Inc., Cary, N.C.), antibiotic drugs (e.g., clindamycin-based antibiotics and antibiotics under the trade designation “AUGMENTIN” from GlaxoSmithKline plc, London, UK), large molecules such as plasmid DNA, and the like. Nanoparticle suspensions with the ability to release drugs over extended periods of time are yet another example of therapeutic material that can suitably be delivered with device 10.

### Examples

The present disclosure is more particularly described in the following examples that are intended as illustrations only, since numerous modifications and variations within the scope of the present disclosure will be apparent to those skilled in the art. Unless otherwise noted, all parts, percentages, and ratios reported in the following examples are on a weight basis, and all reagents used in the examples were obtained, or are available, from the chemical suppliers described below, or may be synthesized by conventional techniques.

Test runs were performed with a device of the present disclosure having a needle assembly with a pair of concentric needles. The inner needle had an inner diameter (corresponding to diameter 88, shown in FIG. 3) of 0.11 millimeters, the annular lumen region between the outer needle and the inner needle (corresponding to annular distance 90, shown in FIG. 3) was 0.02 millimeters, and the offset distance between the needle tips (corresponding to offset distance 86, shown in FIG. 3) was 0.3 millimeters.

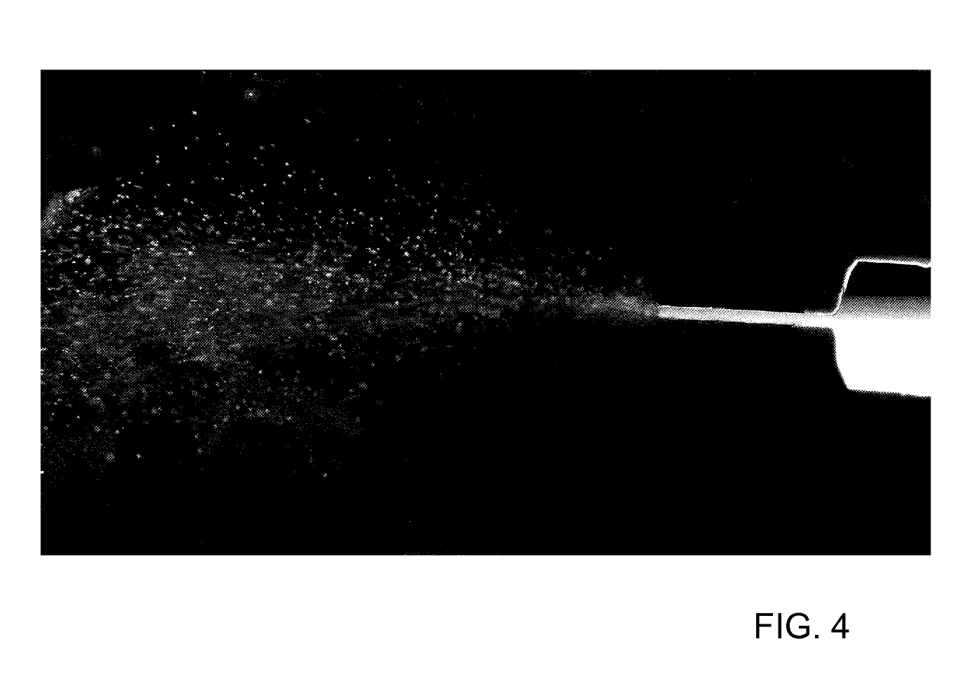

Gas was introduced to the base portion of the device at pressures ranging from about 10 pounds/square-inch to about 60 pounds/square-inch, and liquid drugs were introduced to the drug port to generate multi-phase mixtures at the distal end of the needle assembly. FIG. 4 illustrates a multi-phase mixture being ejected from the distal end of the needle assembly as a conical spray. As shown in FIG. 4, the ejected spray included droplets of the liquid dispersed and suspended in the gas flow. As such, for the tested gas flow rates, the combination of the tip offset distance, the inner diameter of the inner needle, and the annular distance between the concentric needles were suitable for generating and dispensing the liquid and gas as a multi-phase mixture to target locations.

Although the present disclosure has been described with reference to preferred embodiments, workers skilled in the art will recognize that changes may be made in form and detail without departing from the spirit and scope of the disclosure.

1. A device for delivering a multi-phase mixture of a composition dispersed in a gas, the device comprising:
an outer needle comprising a hub region and a tip separated along a longitudinal axis, wherein the hub region of the outer needle comprises an inlet opening configured to receive a supply of the composition; and
an inner needle disposed within the outer needle, the inner needle comprising a hub region and a tip separated along the longitudinal axis, wherein the hub region of the inner needle comprises an inlet opening configured to receive a pressurized supply of the gas, and wherein the tip of the inner needle is offset inward from the tip of the outer needle along the longitudinal axis by an offset distance.

2. The device of claim 1, wherein the offset distance ranges from about 0.01 millimeters to about 1.0 millimeter.

3. The device of claim 2, wherein the offset distance ranges from about 0.1 millimeters to about 0.5 millimeters.

4. The device of claim 1, wherein the inner needle has an average inner cross-sectional area ranging from about 0.002 square millimeters to about 0.2 square millimeters.

5. The device of claim 4, wherein the outer needle and the inner needle define a lumen region between the outer needle and the inner needle, the lumen region having an average cross-sectional area ranging from about 0.005 square millimeters to about 0.14 square millimeters.

6. The device of claim 1, wherein the inlet opening of the outer needle is accessible in a direction that is orthogonal to the longitudinal axis.

7. The device of claim 6, wherein the inlet opening of the inner needle is accessible in a direction that is parallel to the longitudinal axis.

8. A device for delivering a multi-phase mixture of a composition dispersed in a gas, the device comprising:
a base component configured to receive a supply of the gas;
a needle assembly connectable to the base component, the needle assembly comprising:
an outer needle comprising an inlet opening adjacent to the base component and configured to receive a supply of the composition, and a tip; and
an inner needle disposed within the outer needle, the inner needle comprising an inlet opening configured to receive the gas from the base component, and a tip, wherein the tip of the inner needle is offset inward from the tip of the outer needle along a longitudinal axis of the needle assembly by an offset distance.

9. The device of claim 8, wherein the base component comprises:
a conduit configured to direct the received supply of the gas to the inlet opening of the inner needle;
a valve coupled to the conduit; and
a switch configured to operate the valve.

10. The device of claim 8, wherein the base component comprises a flow port configured to access the inlet opening of the outer needle to direct the supply of the composition to the outer needle.

11. The device of claim 10, wherein the inlet opening of the outer needle is accessible in a direction that is orthogonal to the longitudinal axis.

12. The device of claim 8, wherein the offset distance ranges from about 0.01 millimeters to about 1.0 millimeter.

13. The device of claim 8, wherein the inner needle has an average inner cross-sectional area ranging from about 0.002 square millimeters to about 0.2 square millimeters.

14. The device of claim 13, wherein the outer needle and the inner needle define a lumen region between the outer needle and the inner needle, the lumen region having an average cross-sectional area ranging from about 0.005 square millimeters to about 0.14 square millimeters.

15. A method for dispensing a multi-phase mixture, the method comprising:
providing a needle assembly having an outer needle and an inner needle disposed within outer needle, the needle assembly having a proximal end and a distal end offset along a longitudinal axis, wherein the outer needle and the inner needle each have a tip at the distal end of the needle assembly,
directing pressurized gas through an inner lumen of the inner needle such that the pressurized gas is emitted from the tip of the inner needle;
generating a vacuum in a lumen region between the outer needle and the inner needle from the emission of the pressurized gas from the tip of the inner needle;
drawing a composition through the lumen region between the outer needle and the inner needle from the generated vacuum;
mixing the drawn composition and the emitted pressurized gas in a dispersion region, which is located along the longitudinal axis between the tip of the inner needle and the tip of the outer needle, to form the multi-phase mixture; and
ejecting the multi-phase mixture from the distal end of the needle assembly.

16. The method of claim 15, and further comprising supplying the composition to a hub region of the outer needle, the hub region being located at an opposing end of the outer needle from the tip of the outer needle along the longitudinal axis.

17. The method of claim 15, wherein the dispersion region between the tip of the inner needle and the tip of the outer needle has an offset distance along the longitudinal axis ranging from about 0.01 millimeters to about 1.0 millimeter.

18. The method of claim 17, wherein the offset distance ranges from about 0.1 millimeters to about 0.5 millimeters.

19. The method of claim 15, wherein the inner needle has an average inner cross-sectional area ranging from about 0.002 square millimeters to about 0.2 square millimeters.

20. The method of claim 19, wherein the lumen region between outer needle and the inner needle has an average cross-sectional area ranging from about 0.005 square millimeters to about 0.14 square millimeters.
